(19) United States
(12) Patent Application Publication
(10) Pub. No.: US 2024/0266038 A1
(43) Pub. Date: Aug. 8, 2024

(54) SYSTEM AND METHOD FOR ENHANCING DENTAL TREATMENT EXPERIENCE

(71) Applicant: Dental Q Inc, La Miranda, CA (US)

(72) Inventors: Bahram Yousefi, Fairfax, VA (US); Babak Yousefi, Torrance, CA (US)

(73) Assignee: Dental Q Inc, La Miranda, CA (US)

(21) Appl. No.: 18/418,578

(22) Filed: Jan. 22, 2024

Related U.S. Application Data
(60) Provisional application No. 63/440,220, filed on Jan. 20, 2023.

(51) Int. Cl. G16H 40/20; A61B 1/24; G16H 50/70

(52) U.S. Cl. CPC G16H 40/20; A61B 1/24; G16H 50/70

(57) ABSTRACT

System and method for matching a patient with a dentist or dental provider according to treatment-specific indicators defined by treatment-specific ontologies for each patient and each treatment provided by each dentist. The ontologies consist of clinical, practice site, customers service concepts, and parameters. The clinical concepts of the ontology are treatment-specific and are used to curate dental providers for each treatment offered. The treatment-specific provider ratings are used to match patients with their desired dental provider, thus enhancing their dental visit experience.

### TECHNICAL FIELD

The subject matter described herein relates to systems and methods in the field of data analytics related to the field of dentistry and medicine for the purpose of improving practice processes for providers and patients.

### BACKGROUND

Dental care used to be simple. For example, when symptoms of tooth decay such as toothache appeared, either the dentist visited the patient's home, or the patient went to the dentist's office. But such a model no longer exists. Home care by dentists stopped many decades ago, and even visits to the dentist's office must now be preceded by a confirmed appointment from the provider and are done with sophisticated tools and equipment. At the dental office, the process is not unified and differs from one office to another. Consequently, patients are facing a perplexing situation, and they worry about how much they must spend on dental services. Meanwhile, dental offices are under increasing pressure to acquire patients in the same amount of time with limited resources such as assistants, chairs, staff, etc.

The pressure began with the increasing number of dental offices, while the number of patients seeking care did not increase comparatively mainly due to high costs and complicated procedures. This trend continued through the 1990s but was exacerbated by patient demands for an increased voice in their care, better access to the dentist, and more information about their dental treatment. As a result of cost-cutting, employer-provided insurances, merging, and reengineering, dentists have been left with the dilemma of either reducing service to their patients or increasing capacity. The effect has been a mutual disenfranchisement of both dentists and patients. This is evidenced by the increasing number of dentists, who are now leaving dentistry to pursue other careers, and by the fact that the ones remaining are suffering from low production and inefficiencies.

Several commercial entities have attempted to assist dentists by creating back-office operations and management solutions. Back-office solutions are defined as those processes applied to the current day-to-day management of a dentist's practice (e.g., eligibility, claims, online practice management, etc.). However, they do not address the dentist's relationship with the patient. Unfortunately, implementation of a back-office service has a high impact on the practice because it requires a significant addition of resources and necessitates System and process modifications in the dental office, including substantial training of staff, integration, and work process changes. Therefore, the back-office approach is tied to what have proven to be insurmountable problems involving additional time, cost, and management expenditures for the practice. Companies exist that sell in-house systems for the back-office space, including dental record vendors and practice office management vendors. Recently, such vendors have been redeveloping their burdensome back-office solutions to deliver them in online non-downloadable format (e.g., SaaS) and compete in the space currently occupied by other vendors. Nevertheless, whether delivered online or in-house, each of these solutions has a high impact on the operation of the dental office, and as a result will face significant hurdles before they can be adopted regardless of cost, delivery method, or the like.

At the same time, we live in an age of information and technology. The Internet provides several services to its users, including the World Wide Web (WWW), which is essentially a collection of files, often referred to as Web pages, in a variety of formats Stored on host computers, often called Web servers. A collection of Web pages published by an organization is typically termed a Web Site, wherein its first or highest-level page is termed a “homepage.” Consequently, Internet technology has spawned a health care industry online to provide healthcare information to both patients and professionals, and for any number of reasons, individuals are turning to the Internet with increasing frequency for expertise regarding diseases, medicines, treatments, alternative oral care products, and even the selection of dentists.

Although most web pages are textual documents described in HTML, CSS, and JavaScript (JS), the pages may also include images, still or moving, audio data, and video data. The key feature of HTML is the ability to define Hypertext Links within the document, which provide access points to other parts of the same document, other Web pages, or other internet facilities. CSS is designed to enable the separation of presentation and content, including layout, colors, and fonts. JavaScript supports event-driven, functional, and imperative programming styles. It has application programming interfaces (APIs) for working with text, dates, regular expressions, standard data structures, and the Document Object Model (DOM). To access the World Wide Web, the user employs software on his/her computer known as a Web “browser.” Commercially available browsers include, for example, Chrome, Microsoft Edge, etc. The browser provides an interface, a local cache, and a set of processes for accessing the Internet, navigating over the myriad sites, communicating with a selected site, including e-mail, error detection and correction, and security facilities. HTTPS messages consist of either request from a user to a server or responses from a server to a user. The user enters the address (Uniform Resource Locator (URL)) of a web page into his/her browser or selects one from a list of previously stored addresses, often referred to as Bookmarks. The URL is a descriptor that specifically defines a type of Internet resource and its location, e.g., the address of the Web Server holding that Web page, which has an address beginning “HTTPS://”. Access to most web pages is unrestricted; however, access can be controlled using passwords and Security restrictions After the Web page address is entered, the Web browser automatically contacts the user's Service provider and issues a request for that Web page. The Web browser then sends a request to the Web server, which response to the request by sending the requested object to the user. In most cases, the object is a plain text (ASCII) document that is written in HTML, CSS, and JS, which the Web browser displays on the user's computer screen.

Even in this age of information technology with WWW advancements there remains a significant need for effective, reliable, and secure communication between patients (potential and existing) and their dentists (or dental professionals), so that, for example, (i) the patient is not left to rely on the information of unknown accuracy from arbitrary service providers on the Internet, (ii), trust in dental practitioners and service to the patient can be restored, and (iii) billing, scheduling, and administrative functions are efficiently facilitated for both dentists and patients. Moreover, there remains a need for restoring communications between dentists and their patients, for enhancing service to patients, and for expanding the capacity of the dental practices, without additional work by the dentist or his/her staff.

In a server-based computing environment, hardware upgrades, application deployment, technical support, and/or data storage can be provided by one or more terminal application servers. As such, the use of server-based computing to support multiple clients can reduce application costs and ensure that applications are accessible by some or all of the supported clients. Server-based computing also provides enhanced security and eliminates the need for a patch through software upgrades to a plurality of different clients. Traditionally, server-based computing has been implemented by way of remote desktop computing or remote application computing.

Software as a Service (SaaS) generally refers to a concept regarding services relating to the delivery and remote access of software applications via the internet. This concept is often referred to as On-Demand Applications or On-Demand Software. Characteristics of SaaS desirably include: (i) Network-based access; (ii) Management of software; (iii) Activities that are managed from central locations rather than at each customer's site, thereby enabling customers to access applications remotely via the Internet; and (iv) Application delivery is usually closer to a one-to-many model (e.g., single instance, multi-tenant architecture) than to a one-to-one model, including architecture, pricing, partnering, and management characteristics.

J2EE Connector Architecture (JCA) generally refers to a standards-based mechanism of accessing legacy systems from Java/J2EE applications that significantly reduces the inherent challenges of legacy system integration. Connector builders (e.g., the Sun One Connector Builder) can provide graphical wizards to guide a developer through the connector development process. They can also provide code generators that create customized code to enable fast, easy integration of connectors with back-end enterprise and legacy systems. Other connector architectures can be used when appropriate in addition to, or place of, this exemplary connector architecture.

Hub and Spoke Architecture generally refers to a software architecture where the central software framework manages and interacts with multiple lightweight software components that are distributed across a network.

Extract, transform and load (ETL) tools generally enable companies to move data from multiple sources, reformat and cleanse it, and load it into another database, a data mart, or a data warehouse for analysis, or on another operating system to support a business process. Such tools generally provide graphical user interfaces to map the source and destination data visually and provide runtime components that can transform according to these maps.

WWW and remote access to software tools (e.g., SaaS) are combined with artificial intelligence, machine learning, deep learning, and knowledge-based systems to address the issue of patient and dentist relationship in this disclosure.

### SUMMARY

The claimed method and apparatus advance the status quo in the field of dentistry. First, patients are provided with precise information about their options. Therefore, patients can be confident about cost and patient experience they are receiving. Second, dentists are provided with actionable information about their services and the ways they can improve patient experience and ultimately increase practice productivity and advance their specialty field in dentistry. Therefore, dentists will have more satisfied patients.

The claimed method uses a novel algorithm designed to associate one or more patient experience categories, each of which has an estimated price range, according to a plurality of inputs provided by a patient through a client interface(s) of the system. The system estimates a price range for a particular service (e.g., orthodontics) based on the client's desired parameters entered through the interface and the corresponding patient experience associated with them. The system generates an output containing one or more service options that best match the patient's estimated cost and desired patient experience.

In this system, for example, the concept of patient experience has at least the following three components: 1) clinical components, 2) site components and 3) customer service components provided by patients and each practice staff.

The clinical components are largely provided by a dentist and dental assistants or dental hygienists and are significantly influenced by the hands-on expertise of the dentist and the dental assistant. Furthermore, the clinical components may be influenced by the quality of the equipment used in the practice. Due to the intricated nature of the clinical components usually, the patients can't have any assessment of the clinical treatment they receive; thus, the current system and method uses a novel ontology to assess the clinical component for each provider in the provider networks, a novel ontology is used to perform the assessment. The ontology used is dynamic and continuously updated to match the current ground truth (e.g., information that is known to be real or true, provided by direct observation and measurement as opposed to information provided by inference) of the specialty. For example, if a new technique or tool is introduced that changes the clinical quality in a meaningful way.

The site components include everything from decoration, paint colors, smells, sound systems, equipment, and so forth; all of which relate to the practice site (i.e., structural exterior and interior features of the location). All the site component concepts are captured in the claim ontology for scoring, matching, and comparison. The practice staff can provide a courteous encounter and handle the patient's case correctly and efficiently. Usually, the patient perception of the patient experience is weighted by the site components and the customer service components. As a result, in the modern age of the dental industry, clinical components are not valued significantly by the patients and in turn, dentists are shifting their focus from clinical components to the site components and the customer service components.

External platforms that exacerbate the shift are the popular online reputation and ranking platforms such as Google Business, Yelp, and so forth. All these platforms publish reviews and rankings that are not designed to capture or value the clinical components of any dental services. Such online reputation and ranking platforms are focused on the site and the customer service components and are directly publish customer reviews that may not contain actionable insights for a professional business such as dentistry. To this end, in the status quo, there are dental practices that invested a lot in their office decoration, office design, and training staff for upscale customer service but did not invest as much in clinical components, thus the services offered in such practices may be substandard.

To prevent practices from neglecting the quality of their clinical work the current system utilizes a novel scoring and ranking method which factors the quality of the clinical work as well as the site and the customer service offered by the staff. Thus, for example, the penultimate impact of including the clinical components in the assessment of dental service is a more economical treatment for the patients because of the shorter chair time. Ultimately, this means more productive practices and more satisfied patients.

### BRIEF DESCRIPTION OF DRAWINGS

The present system and method are illustrated by way of example and not limited by the figures of the accompanying drawings in which like references indicate similar elements.

FIG. 1 illustrates one embodiment of a network diagram, including external partners and services.

FIG. 2 illustrates different modules of the system, according to one embodiment.

FIG. 3 illustrates an exemplary method for patient journey.

FIG. 4 illustrates an exemplary method for provider (e.g., dentist) journey.

FIG. 5 illustrates an exemplary ontology for providers in the field of orthodontics, e.g., providers in other specialties (e.g., implant) may have different ontologies.

FIG. 6 illustrates an exemplary ontology for patients in the field of orthodontics, e.g., patients in other specialties (e.g., implant) may have different ontologies.

### DETAILED DESCRIPTION

Various embodiments of the present system and method are now described in reference to the drawings. In the following description, for purposes of explanation, numerous specific details are set forth to provide a thorough understanding of one or more implementations of the present invention. It will be evident, however, that the present invention may be practiced without these specific details. In other instances, well-known structures and devices are shown in block diagram form to facilitate describing the present invention.

As used in this application, the terms “module” and “system” are intended to refer to a computer-related entity, either hardware, a combination of hardware and software, software, or software in execution. For example, a module may be but is not limited to being, a process running on a processor, a processor, an object, an executable, a thread of execution, a program, and/or a computer. By way of illustration, both an application running on a server and the server can be a module. One or more modules may reside within a process and/or thread of execution and a module may be localized on one computer and/or distributed between two or more computers.

The present system and method may also be illustrated as a flow chart of the process of the system and method. While, for simplicity of explanation, the one or more methodologies shown in the form of a flow chart are described as a series of acts, it is to be understood and appreciated that the present invention is not limited by the order of acts, as some acts may following the present invention, occur in a different order and/or concurrent with other acts from that shown and described herein. For example, those skilled in the art will understand and appreciate that a methodology could alternatively be represented as a series of interrelated states or events, such as in a state diagram. Moreover, not all illustrated acts may be required to implement a methodology of the present system and method.

In various embodiments disclosed herein, the term “dentist”, “doctor” or “provider’ is used to identify a dentist or other dental/hygiene provider administering patient care, as well as members of his/her staff that assist in providing such care or are responsible for maintaining the provider's scheduling calendar and/or patient records.

Further, in various embodiments disclosed herein, “practice group”, “provider group”, “dental group”, or “dental support (service) organization (DSO)”, may be used to refer to any entity linking a group of providers through shared facilities, resources, services, or referral agreements. This may include but should not be limited to integrated multi-practice dental service providers, insurance networks, membership networks, dental groups, and multi-dentist practices.

As used herein “patient” or “client” is used to refer to an existing patient, or a prospective (potentially new) patient, of a dental provider, dentist, or practice group.

In one or more embodiments of the system described herein, a “User Interface Module” is a centralized service provider that provides a wired or wireless network-based service to the practice groups (e.g., dental groups and DSOs), solo practices, remote sites (e.g., department stores, shopping malls, drug stores, and so forth, where a pertinent kiosk or terminal is installed), and patients. In addition, the user interface module provides the communication channel, protocol, and interface for connection to other stakeholders (e.g., payments systems, back-office services, membership services, insurance service brokers, and external partners/brokers). For example, in the user interface module, the server may provide an application or web-based data processing service and interface to a computer, server, or other wired or wireless communication devices (e.g., mobile phone, tablet computer, wearable devices, etc.) of one or more practice groups, solo practitioners, and patients.

### System Architecture

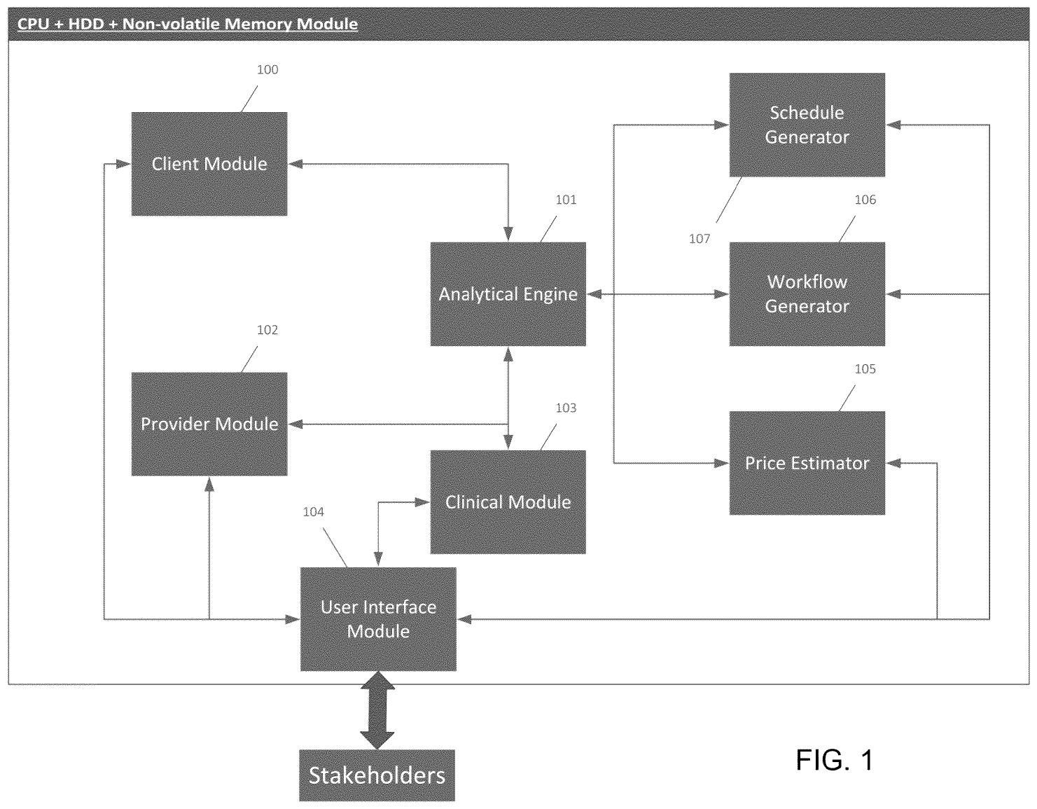

FIG. 1 illustrates a high-level system architecture, according to one embodiment. In this embodiment, for example, user interface module 104 communicates with each of a plurality of patients by rendering a patient interface to facilitate the acquisition and/or collection of patient information from the patients and for completion or partial completion of patient information forms and/or questionnaires. The acquired information is preprocessed and securely stored in client module 100. The information and the pertinent forms and questionnaires may be tailored according to treatment of interest. For example, the information for orthodontic treatment would be specific to orthodontics and may be completely different from endodontics. To this end, treatment-specific ontologies may be developed and recorded in the client module 100 and used by the user interface module 104 to render the patient interface specific to the treatment of interest.

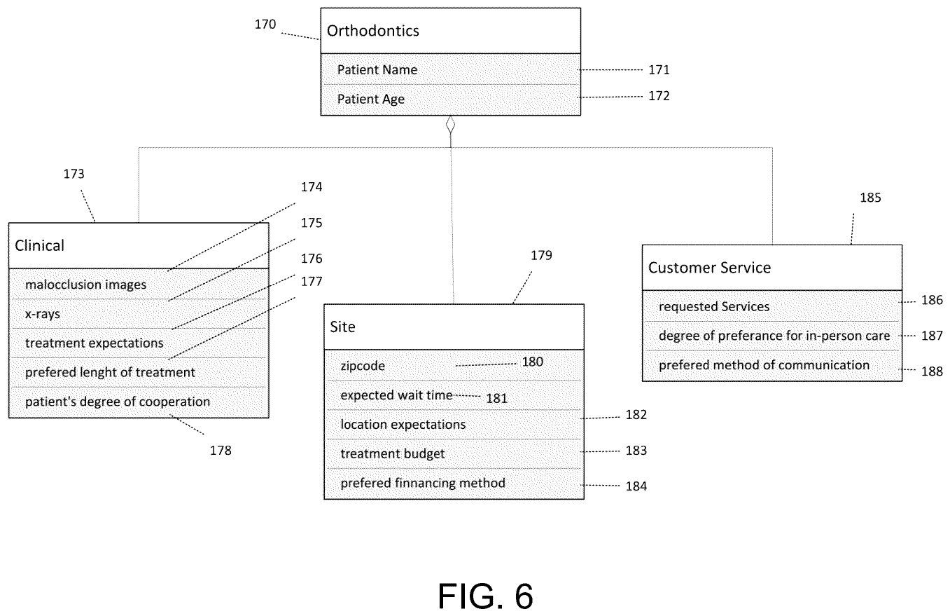

The treatment-specific ontologies, as illustrated in FIG. 6, are developed by subject matter experts, artificial intelligence algorithms, data mining, or a combination of such methods and updated (automatically or user input) as needed to match the ground truth about the specific treatment.

Furthermore, the user interface module 104 is configured to communicate with a plurality of dental groups and/or individual dental service providers (i.e., solo practices) rendering a provider interface to facilitate the gathering of provider information from the dental groups or individual dental service providers and for the completion or partial completion of practice information forms, questionnaires, test results, certificates, or in-person audits. The acquired information is preprocessed and securely stored in provider module 102. The information and practice-related data pieces may be tailored according to the specific treatment. For example, the information for orthodontic treatment may be specific to orthodontics and may be different from endodontic treatment. To this end, treatment-specific ontologies are developed and recorded in the provider module and used by the user interface module to render the patient interface specific to the treatment of interest.

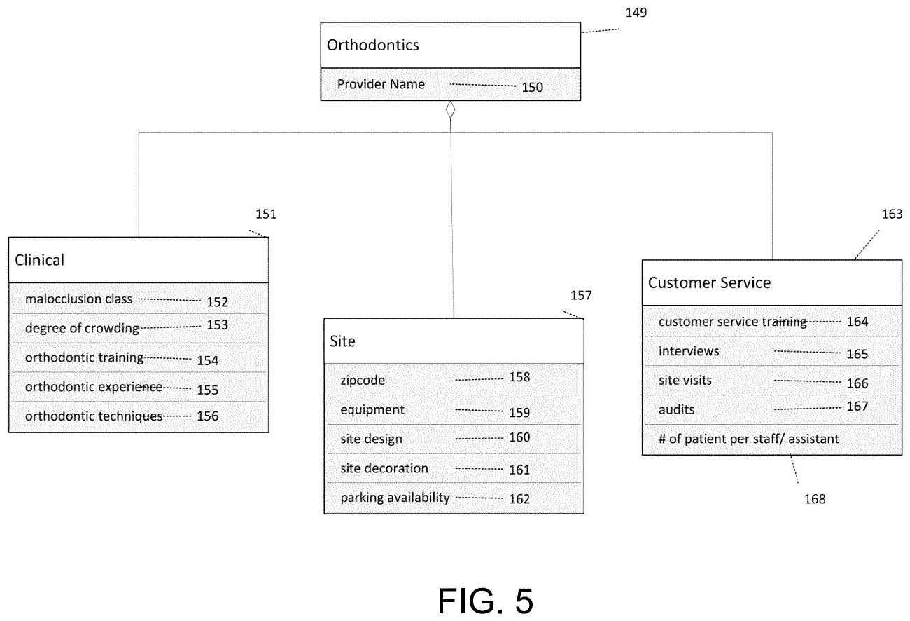

The treatment-specific ontologies, as illustrated in FIG. 5, are developed by subject matter experts, artificial intelligence algorithms, data mining, or a combination of such methods and updated as needed to match the ground truth about the specific treatment. The ontologies for patients and providers may be different, but they are aligned such that they can match and compare against each other. One novel feature of both patient and practice ontologies is that it includes concepts for clinical aspects of the specific treatment. For example, an orthodontic treatment-specific ontology for a provider may be designed to include one or more concepts such as malocclusion class 152, degree of crowding 153, orthodontic training 154, orthodontic experience 155 and orthodontic techniques 156, and so forth. Furthermore, the ontologies may be designed to include concepts about the practice site 157, including, but not limited to, zip code 158, equipment 159, site design 160, site decoration 161, parking availability 162, and so forth. The ontologies may also be designed to include concepts about the customer service 163 provided by the practice doctors and staff, including, but not limited to required and mandatory training results 164, interview results 165, results from site visits 166, audit results 167, the ratio of the patients to staff 168, and so forth.

The user interface module 104 may use the treatment-specific ontologies and a database of questions and information forms to render interfaces for soliciting information from the stakeholders, which includes patients, dental offices, dentists, etc.

In the embodiment shown in FIG. 1, for example, the user interface module 104 uses Schedule Generator module 107, Workflow Generator module 106, Price Estimator module 105, Clinical module 100, and Analytical Engine 101 to render interfaces to display results and notifications to the stakeholders, which includes patients, dental offices, dentists, etc. One of ordinary skill in the art may design the system to use any one or more of these modules to render such interfaces.

Client module 100 is configured to securely store patient information, ontologies, and a database or database of questions and information forms. The data on the client module 100 can be searched and utilized to improve the quality of the ontologies to fit the state-of-the-art in the specific field. Furthermore, other modules such as user interface module 104 and analytical engine 101 can access the client module 100 to obtain information needed to render interfaces and analyze the inputs.

Provider module 102 is configured to securely store provider information, ontologies, and a database or set of databases of questions and information forms. The data on the provider module 102 can be searched and utilized to improve the quality of the ontologies to fit the state-of-the-art in the specific field. Furthermore, other modules such as user the interface module 104 and the analytical engine 101 can access the provider module 102 to obtain information needed to render interfaces and analyze the inputs.

The client module 100 and the provider module 104 are configured to provide data for the analytical engine module 101 to perform various analyses, also they provide data for the user interface module 104 to render user interfaces for stakeholders.

The analytical engine module 101 is configured to compare the patient instance of the treatment ontology (e.g., orthodontics treatment) with the available practitioner's instances of the ontology of the same treatment within a specified radius mile to determine the closest matching providers. For example, the engine 101 uses an Artificial Intelligence (AI) technique to compare the two ontologies (e.g., the patient instance of the treatment ontology and the available practitioner's instances of the ontology of the same treatment) and calculates a similarity metric using methods surveyed by E. Thiéblin, O. Haemmerlé, N. Hernandez, and C. Trojahn, in “Survey on complex ontology matching,” the entire disclosure of which is incorporated herein by reference, except to the extent that the incorporated material is inconsistent with the express disclosure herein, in which case the language in this disclosure controls. The similarity metric is generated by comparing two ontologies and calculating an aggregate score that estimates how similar the two ontologies are. The engine uses multiple models to match the patient and provider ontologies and then either selects or aggregates the results from the models to increase the reliability. This technique is published by K. B. Laskey et al., “Modeling inference enterprises using multiple interoperating models,” the entire disclosure of which is incorporated herein by reference, except to the extent that the incorporated material is inconsistent with the express disclosure herein, in which case the language in this disclosure controls. For example, the novel contribution of this invention is including the clinical components and creating a multifaceted ontology that has clinical concepts specific to the dental treatment and then using the AI-enabled ontology matching method to find a few close matches to the desired ontology (i.e., patient's ontology). This method finds at least one close match that meets the patient's preferences such as distance, amenities, clinical ranking, and so forth. The matched provider(s) is presented to the patient via the user interface module 104. In addition, the analytical engine 101 is configured to perform all calculations for clinical module 103, schedule generator module 107, workflow generator module 106, and price estimator module 105.

The analytical engine module 101 may use various similarity measurements one of which is a two-layer methodology base on the lexical and conceptual levels of the two ontologies. The similarity score is calculated using

$$\overline{RO} = \frac{1}{ℒ} {\sum }_{\vert L ∈ ℒ \vert} RO ( L , {On}_{1} , {On}_{2} )$$

where L is the lexical (set of terms) mapping of the ontologies and O is the core ontology (a tuple of concepts, relationships, statements, and terms). The similarity measures are then ranked to find a close score that satisfies all the clinical and non-clinical preferences. The term close is constantly updated as the patients and practitioners onboard the system, however especially initially it is possible that no close score could be found (e.g., the rankings are continuously updated). The initial goal (threshold) for a close score is a score that matches the location proximity and clinical preferences at the minimum.

The clinical module 103 analyzes the data entered by a patient or one or more providers to determine a proper course of action for the specific treatment. For example, for orthodontic treatment, the 2D images of a dental arch of a patient taken from different angles are sent to the analytical engine module 101.

The analytical engine 101 uses deep learning or machine learning to construct a full arch representation from the received images. The complete arch representation in either 2-dimensional or 3-dimensional pictures is presented to subject matter experts (e.g., orthodontists). Also, a deep learning analysis may be performed on the reconstructed images to provide decision support to the subject matter experts. A proper course of action is entered by the subject matter expert through the user interface module 104. The course of action will include all the necessary details required to estimate a price range and find one or more matching experiences. For example, a sample course of action for orthodontics treatment may include the complexity of the case, type of malocclusion, estimated duration of treatment, and applicable treatments for the patient. This process is fully monitored by a subject matter expert (e.g., orthodontist) through the user interface module 104. However, this process can be done fully automated using artificial intelligence methods. Clinical module 104, can process various images, x-rays, and textual and visual clues relevant to the specific treatment to produce the proper course of action for the treatment. A proper course of action is generated by analysis engine module 101, schedule generator module 107, the workflow generator module 106, price estimator 105, and clinical module 103 and presented to the patient by the user interface module 104. A course of action, for example, can help the patient with putting forth the pricing of different treatment options, estimating the length of treatment, and scheduling appointments, and any necessary workflow options such as filling the forms before the appointments and financial and payments for the visit, and so forth. As a result, patients are well informed and prepared for their appointment, which ultimately enhances the patient experience.

The schedule generator module 107 will provide booking option(s) to the patient and confirm the booked appointment(s) with the provider. Also, in treatments such as orthodontics and implant where multiple appointments may be needed in specific intervals this module will help the patient with booking their next appointment and confirm the same with the provider's office.

The workflow generator module 106 is designed to handle all actions needed on the patient side. The actions can include, for example, filling in the forms and consent letters, getting information about their visit, the financial options, and necessary payments, and so forth. This module will handle all the financing and discount options available to patients, so patient's concerns regarding treatment costs and financing options are addressed transparently before each visit. The financing options are treatment specific, for example, orthodontic treatment may include installment options for the duration of the treatment, as well as discount program membership for the duration of the treatment and guarantee program membership for coverage and discounts after the treatment is completed. It may include guarantee programs designed to cover certain relapses and offer any repairs and redo at a discounted price. Further, the workflow generator module 106 may be configured to monitor or track the teeth and arch movements during the treatment and after the completion of the treatment plan using the images and/or x-rays submitted by either a patient or a provider. The reconstructed images (from user-submitted images and x-rays) may be used to track the changes in the arch and individual tooth movements. The tracking may be done with a smartphone application, wearable device application, kiosks installed in malls, shopping centers, retail stores, pharmacy stores, etc. The orthodontic tracking can give a provider the ability to remotely track (e.g., telehealth) the patient's progress rather than scheduling a visit, thus it can save time for both patients and providers.

The price estimator module 105 is configured to leverage the data provided by the patient through the user interface module 104, the option(s) matched for the user by the analytical engine 101, and the assessments entered by subject matter experts (e.g., orthodontists) and produced by artificial intelligence to calculate a price or price range that helps the patient make a better-informed decision about his/her treatment options. The price ranges are presented through the user interface module 104.

The system may be designed so that both the patient and the user can provide feedback about their experiences through the user interface module 104. The patient reviews may be parsed according to the patient and provider ontologies using artificial intelligence techniques for text, voice, video mining. The parsed data may be used to produce a constructive and actionable report for the provider to improve their practice.

A reputation score may be calculated or determined that is influenced by the patients' reviews. The reputation score may be private or public. Unlike, current generic online reputation platforms, the current system and method uses patient reviews as one factor for calculating the reputation score. There are other factors and contributors such as clinical components, site components, and customer service components. One advantage of utilizing this aggregation method to calculate the reputation score is that it helps new providers and practices gain reputation and prevent malicious exploits from damaging a practice reputation. The patient reviews may even help providers improve their services.

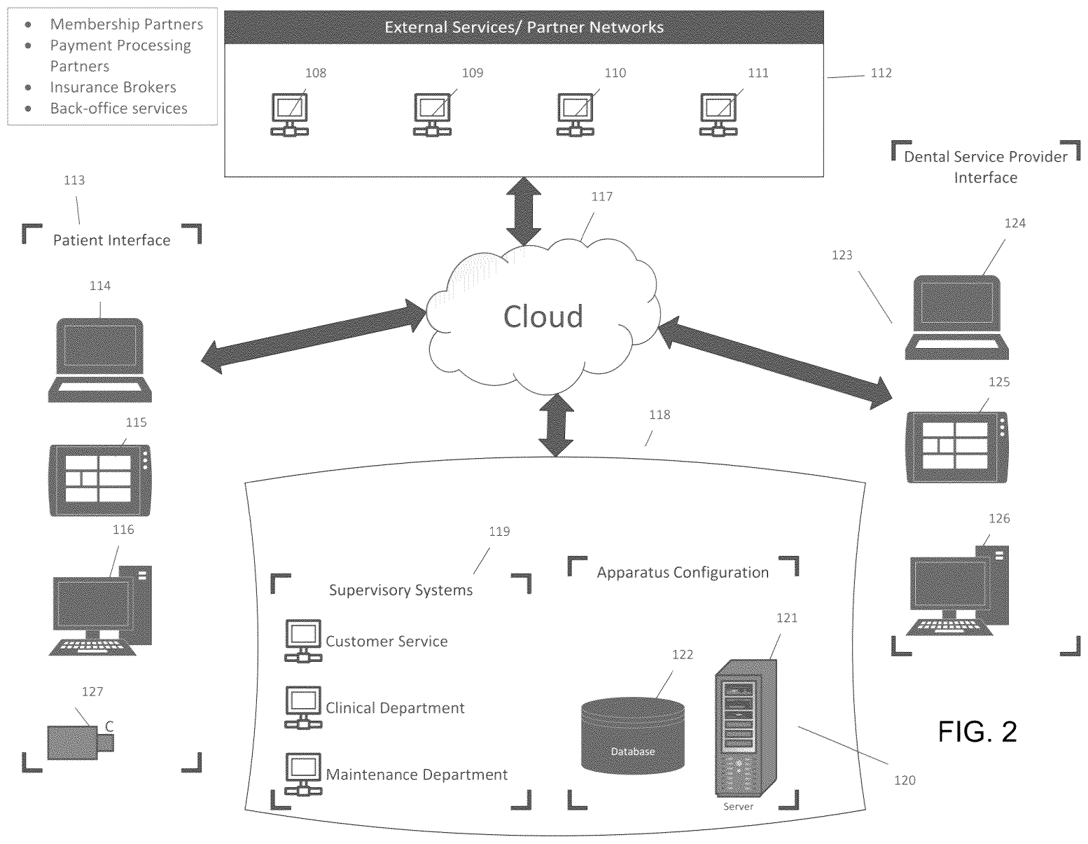

FIG. 2 illustrates one embodiment of a network diagram of the invention and all the stakeholders and systems that may interact with it. Apparatus 118 is configured as server 121 executing the methods of the system described herein and includes database 122 that securely stores information required for the system. The system has a connection to supervisory systems 119 configured to monitor execution of different methods and provide inputs as needed. For example, subject matter experts may review the dental arch images and input their diagnostics through the terminal rendered by the user interface module 104. The supervisory systems 119 may include one or more of customer service, clinical department, and maintenance department. For example, the customer service system may be utilized to handle patient calls and electronic communications, the clinical department may be utilized to review the entered data and suggestions obtained from the artificial intelligence methods to produce the course of action for the treatment of the patient, and the maintenance department may be utilized to monitor the server and systems status and performance. The devices through which a patient or a provider can access the system 118 are computers, laptops, tablets, wearable devices, camera-enabled devices, and kiosks, etc. External systems that may interact with the system 118 include, for example, payment processing gateways, discount and membership program vendors, insurance and clearance houses, vendors providing back-office services.

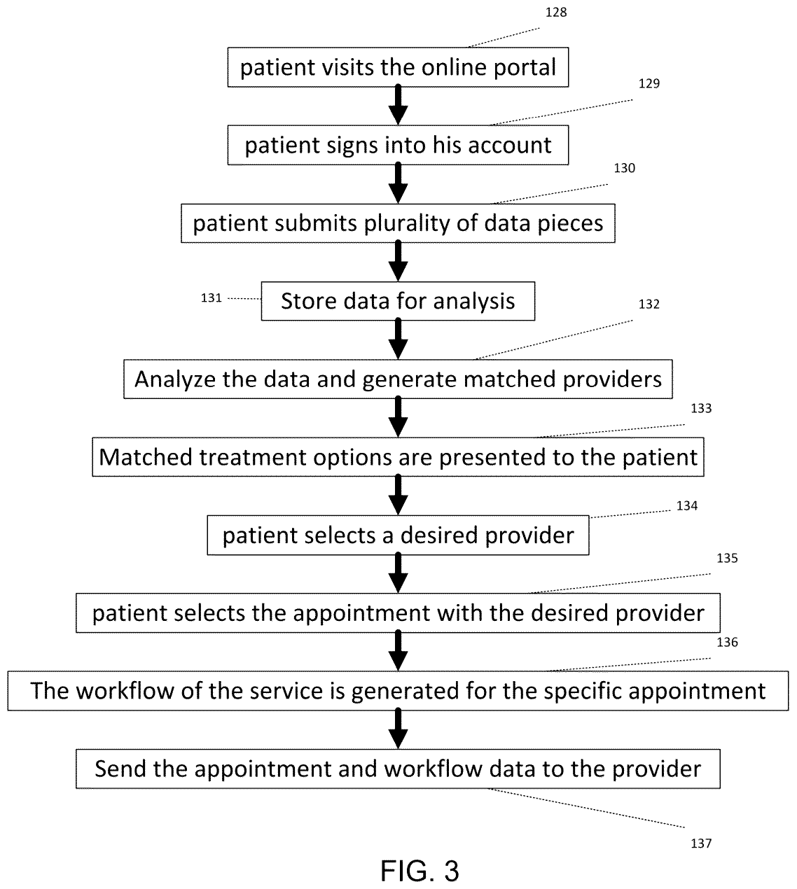

FIG. 3 illustrates an example of a patient journey on the system's online portal. In this example, a patient visits online portal 128, where he/she can create an account (secured) and sign in to his/her account 129. One or more questions are presented to the patient, answers of which provide a plurality of data pieces 130. The data is stored in the client module 100. The data is analyzed by the analytical engine module 101 to find matching providers 132 that meet the patient's requirements by using the highest aggregate scores that are calculated by comparing a patient ontology instance and providers ontology instances in the pre-defined geographic proximity. The matched provider(s) is presented to the patient 133 and then the patient can select his/her desired provider 134. The patient can make an appointment on the online portal 135. A workflow and schedule for the patient are created 136 and sent to the provider for confirmation 137.

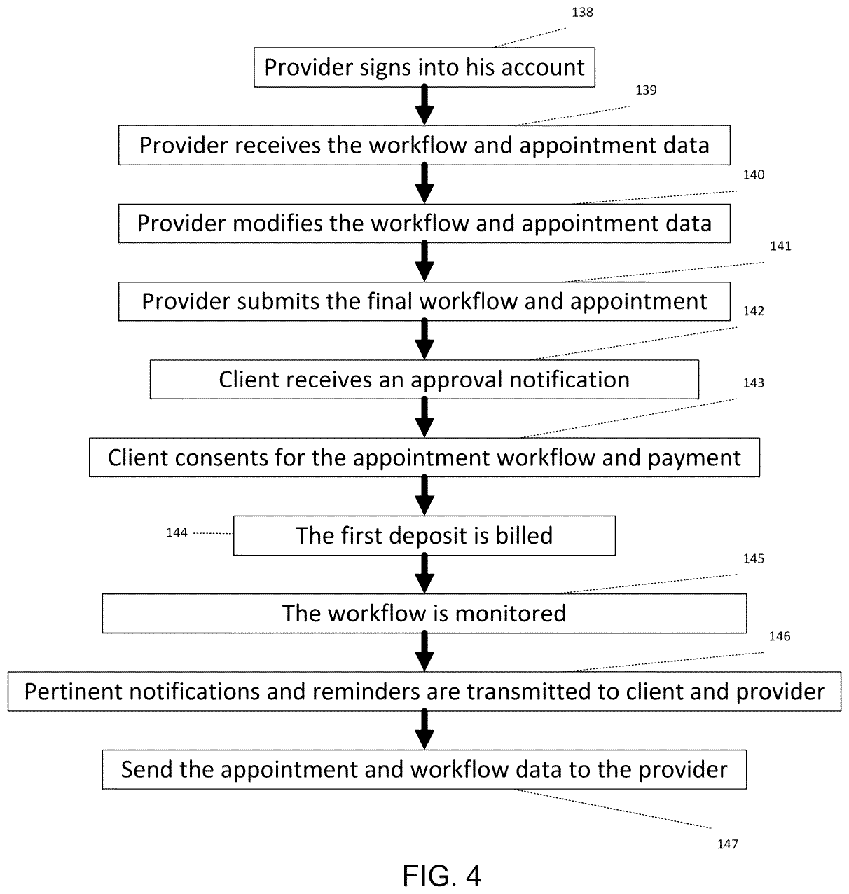

FIG. 4 illustrates an example of a provider journey on the system's online portal. A provider visits the online portal and signs in his/her account 138. The provider then can receive the booked appointment data 139 and modify it 140 as needed. The provider then confirms/approves appointment 141 which in turn is sent to patient 142. The patient then consents to the appointment and corresponding fees 143. The fees may be financed and paid in installments; thus, the pertinent installment is billed 144. The appointment workflow is then activated 145 to guide the patient for any action needed to be performed or completed before the practice visit and both patient and provider are notified about any required actions 146. The consented appointment data will be sent to provider 147.

FIG. 5 illustrates an example provider ontology. The example is based on the specific orthodontic treatment and has all the concepts pertinent to the provider of the specific treatment. As discussed above, the ontology has at least the following three main components: 1) clinical components 151, 2) site components 157, and 3) customer service components 163. Each of these components and the respective values are stored in the provider module 102. The components are defined and entered into the system by subject matter experts. Furthermore, machine learning and deep learning may be used to mine resources and articles to create and update the ontologies. To adhere to the nuances of each treatment, each treatment will have a separate ontology.

FIG. 6 illustrates an example of patient ontology. The example is based on the specific orthodontic treatment and has all the components pertinent to the patient seeking the specific treatment. As mentioned above, the ontology has at least the following three components: 1) clinical components 173, 2) site components 179, and 3) customer service components 185. Each of these components and the respective values are stored in the client module 100. The components are defined and entered into the system by subject matter experts. Furthermore, machine learning and deep learning may be used to mine resources and articles to create and update the components. To adhere to the nuances of each treatment, each treatment will have a separate ontology.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the methods and embodiments described herein. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc, laser disc, optical disc, digital versatile disc, floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present subject matter, e.g., for applications suitable for public and private sectors. Various modifications to these embodiments will be readily apparent to those skilled in the art, and the generic principles defined herein may be applied to other embodiments without departing from the spirit or scope of the subject matter. Thus, the present subject matter is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the claims in the non-provisional application claiming priority to this provisional application.

We claim:

1. A computational apparatus configured to:
receive a plurality of data pieces from the patient;
generate a treatment-specific ontology instance for the data entries of each patient, concepts of which comprise: clinical components, site components, and customer service components;
estimate a price range for the generated patient ontology;
receive a plurality of data pieces from the dental service provider;
generate a treatment specific ontology for the particular data entries of the dental service provider, concepts of which comprise (i) clinical components, (ii) site components, and (iii) customer service components;
estimate a price range for the generated provided ontology;
compare the patient's ontology with a set of providers' ontologies within a defined geographical area;
compare the estimated cost of the treatment with a set of providers' costs within the defined geographical area;
calculate an aggregated score for matching patient ontology and price range to providers' ontologies and estimated costs;
select a predefined number of best-matching providers with the highest aggregated scores within the defined geographical area;
display the best matching dental service providers to the patient;
enable the patient to book an appointment with the desired provider;
inform the provider about the booked appointments.

2. The apparatus of claim 1, wherein price estimates are produced by a decision support system that uses deep learning.

3. The apparatus of claim 1, wherein the patient can access an online portal to book the appointment via a kiosk.

4. The apparatus of claim 1, wherein a tracking app sends a patient's progress reports to the desired provider.

5. The apparatus of claim 1, wherein clinical decisions are made using artificial intelligence techniques.

6. The apparatus of claim 1, wherein online payment services are provided to the patient.

7. The apparatus of claim 1, wherein a patient's ontology is analyzed using artificial intelligence techniques to find treatment options.

8. The apparatus of claim 1, wherein a provider's ontology is analyzed using artificial intelligence techniques to estimate a reputation score for the specific treatment.

9. The apparatus of claim 1, wherein the best-matching providers are identified using multiple ontology matching models to increase the reliability of the method.

10. The apparatus of claim 4, wherein the tracking app takes pictures of a dental arch using a camera-enabled device.

11. The apparatus of claim 4, wherein the tracking app takes pictures of the dental arch using a camera-enabled kiosk.
